(19) United States
(12) Patent Application Publication
(10) Pub. No.: US 2012/0321763 A1
(43) Pub. Date: Dec. 20, 2012

(54) Doneness indicator for cooking meats

(75) Inventor: Douglas C. Arndt, Glendora, CA (US)

(73) Assignee: Innovative Product Development

(21) Appl. No.: 13/134,858

(22) Filed: Jun. 20, 2011

(51) Int. Cl. G01N 33/12; A23L 1/01

(52) U.S. Cl. 426/233

(57) ABSTRACT

A method of ensuring that meat has been adequately cooked to doneness, wherein a substrate, such as a foil, film, fabric, laminate, label, card, or paper, having a thermo-sensitive color-forming coating thereon, is placed onto meat pieces such that cooking causes the coating to change color at a predetermined temperate. Said color-forming coating is comprised of ingredients generally recognized as safe as food additives or as food colorants by the United States Food and Drug Administration (FDA), or as materials FDA-approved for food contact.

### FIELD OF THE INVENTION

The present invention relates directly to preparing foods and most specifically to cooking raw meat products.

### BACKGROUND OF THE INVENTION

Meat that is under-cooked may be hazardous due to the presence of active, pathogenic bacteria such as e-coli. To kill harmful bacteria one must be certain to cook the meat piece until its internal temperature reaches at least about 165 degrees F. A thermometer may be inserted into the piece of meat to check temperature, but this repeated task can be inconvenient and burdensome, especially when hundreds of meat items are prepared daily as in a restaurant. The use of a thermometer also produces the risk of transferring harmful bacteria from a contaminated piece of meat to a good piece of meat.

Another problem is that of knowing when the interior of the meat item has reached the desired level of cooking, since one cannot actually see into the center of the meat without first slicing it open. Patrons of restaurants typically request that their meat be cooked a certain way, as for example, “medium rare” or “well done”. This task can be burdensome to even experienced cooks, and it is not uncommon for meat to be sent back through the restaurant staff to be cooked again.

Thermochromic coatings or ink marks applied directly to the surface of meats may impart unwanted tastes, textures, and appearances, and besides these things, thermochromic inks are not acceptable to the FDA as food or color additives due to their potential toxicity or carcinogenicity. Thermochromic materials also revert back to their original color after they cool and so they are not suitable for double-checking meats which have removed from the cooking process.

Therefore, a need exists for a method that conveniently and safely identifies food that has been cooked to the desired level of doneness.

### SUMMARY OF THE INVENTION

The present invention provides a substrate which may be coated or printed with a thermo-sensitive chemical composition that produces a color at a specified temperature, and this temperature correlates with the internal temperature of the meat piece as it is being cooked. The substrate may be composed of any material suitable for food contact and it may be converted into sheets, labels, disks, or cards which can be placed over one planar side of the meat piece, such as a meat patty, or it may be converted into bands which can be secured around curved meat pieces, such as sausage links. The substrate is located on the meat item such that the thermo-sensitive coating does not contact the cooking surface, but preferably, it is positioned on the side of the meat opposite the cooking surface. As heat rises up through the meat's interior to the thermo-sensitive coating on the substrate, there is no color formation until the pre-determined temperature has been reached. Once the color is formed, the color may remain indefinitely as proof that the meat has been properly prepared.

There are numerous combinations of food additives that can form intense colors. For example, ferric compounds react with gallic acid and its derivatives to form a black color, or with vanillan and other phenolic compounds to form colors ranging from brown, red, orange, grey, blue, and violet. Another example is sodium ferro-cyanide, which can produce a range of colors from white, yellow, green, red, and most notably, Prussian blue, depending on the metal salt employed as the co-reactant. Color changes can also be accomplished using FDA-approved colorants with food additives, wherein the additives affect the color by means of pH or oxidation-reduction. The color formation is controlled by thermo-sensitive materials that melt at the desired temperature. Many approved food additives suitable for this role include waxes, butters, fatty acids, fatty alcohols, and fatty esters. These ingredients are formulated into coatings that are applied to the substrate of choice.

The substrate can be made of metal foil, paper, plastic, or fabric, depending on how it is intended to be in contact with the meat, and it may also be a laminate constructed from these materials and others to best suit the type of meat. The substrate can be coated with a varnish to protect it from grease and juices and to minimize curling. It may also be perforated to allow steam to escape. Food-grade adhesives may also be applied so that the substrate is in the form of a label. The substrate may be printed with information, artwork, or any graphics in general. Numerous modifications are possible to those skilled in the art of food packaging. The thermo-sensitive coating is printed onto the side of the substrate that does not contact the meat, which is the side open to the air and visible to the cook.

Common “fax” paper is most convenient as a substrate material in that a thermo-sensitive color-forming coating is already present. Numerous grades of “fax” paper are available that are FDA-approved for incidental food contact. Another convenient aspect to “fax” paper, or thermal printer paper, is that coatings are commercially available that activate at various temperatures ranging from about 120 degrees F. to about 180 degrees F. or higher. This allows for the construction of labels that can indicate whether the meat is cooked rare, medium rare, well done, or to the point where all harmful bacteria have been destroyed. Thermal printer paper and card stocks are also printable using standard inks and they can be laminated or coated as described previously. Another useful aspect of thermal paper is that it may be printed with chemical activators for precise control of the color-forming reaction in regards to its initiation temperature. Such activators can be found on the GRAS list and include transition metal salts, propyl gallate, glycols, fatty acid esters of glycols, and ethoxylated fatty alcohols.

It is understood by those skilled in the art that numerous modifications to and equivalents of this embodiment can be made without departing from the spirit and the scope of this invention.

What is claimed:

1. The method of cooking food, such as meat, wherein a substrate having one side coated with a thermo-sensitive color forming composition and the other side substantially contacting the surface the food, so that an indelible or semi-permanent record is created when the food is cooked to a predetermined temperature.

2. The method of claim 1 wherein the thermo-sensitive color-forming composition is comprised of food additives and/or color additives generally recognized as safe by the United States Food and Drug Administration (FDA).

3. The method of claim 1 wherein the substrate having the thermo-sensitive color-forming coating thereon is thermal printer paper.

4. The method of claim 1 wherein the substrate having the thermo-sensitive color-forming coating is thermal printer paper that has been printed on its reactive side with additional activators.

5. The method of claim 3 wherein the thermal printer paper is FDA-approved for food contact

6. The method of claim 1 wherein the thermal composition activates within the range of about 120 degrees F. to about 250 degrees F.

7. The method of claim 1 wherein the thermo-sensitive composition activates within the range of about 140 degrees F. to about 190 degrees F.

8. The method of claim 1 wherein the thermo-sensitive composition activates at about 165 degrees F.
